(19) United States
(12) Patent Application Publication
(10) Pub. No.: US 2010/0093073 A1
(43) Pub. Date: Apr. 15, 2010

(54) BIO-REACTOR

(76) Inventors: Ursula Erhardt, Berlin (DE); Christoph Erhardt, Berlin (DE)

Correspondence Address: MAYER & WILLIAMS PC, 251 NORTH AVENUE WEST, 2ND FLOOR, WESTFIELD, NJ, 07090, US

(21) Appl. No.: 11/935,390

(22) Filed: Nov. 5, 2007

Related U.S. Application Data
(63) Continuation of application No. 10/485,603, filed on Jan. 11, 2005, now abandoned.

(51) Int. Cl. C12M 3/00

(52) U.S. Cl. 435/289.1

(57) ABSTRACT

A bio-reactor for cultivating cells, comprising a culture vessel, at least one gas supply and/or liquid feed in addition to supply devices for gases and/or liquids enabling gases or liquids to be fed to the culture vessel. A mixture device, especially a Venturi nozzle is arranged between the supply device and gas supply or liquid feed in order to mix gas and/or liquid from the gas supply or liquid feed.

### STATEMENT OF RELATED APPLICATION

This is a continuation of U.S. patent application Ser. No. 10/485,603, filed Jan. 11, 2005, entitled “Bio-Reactor,” which is a 371 of PCT Application PCT/DE01/02902, filed Jul. 31, 2001, entitled “Bio-Reactor”. Both of the prior applications are incorporated by reference in their entireties herein.

### FIELD OF THE INVENTION

The subject matter of the invention is a method and a device permitting a quantitative production of gas/gas, gas/liquid or liquid/liquid mixtures by a defined supply of the component(s) to be dosed to a carrier medium and thus a precise, quantitative dosage of a single component or a mixture to culture vessels for biological or biochemical reactions.

### PRIOR ART

Culture vessels for biological or biochemical reactions, as far as they are not fermentation systems in a scale larger than 1,000 ml, are today usually neither aerated nor do they have suitable dosing devices. The reason for this is that the technical and economical expenses for these regulation systems according to the state of the art are very high and become technically unreasonable with progressing miniaturization of the culture vessels. The prior art with regard to the present invention known from practice is now presented with reference to various quantitative dosing modules:

### Gas Dosage:

A quantitative gas dosage takes place at a constant inlet pressure by mechanical flowmeters, which are regulated with needle valves to the desired gas flow. Further, there exist electronic mass flow controllers, which automatically regulate the gas flow by a regulator unit and electric adjusting orifices. The regulated gas flow may be in orders of magnitude between ml gas/h and m³ gas/h. Biological or biochemical culture vessels are supplied in each of their applications by its respective gas dosage section. At the outlet opening toward the culture vessel, pearl-type ejectors may be provided. (Braun Biotech International GmbH, bio-reactors series BIOSTAR A, B, MD, Q, D, U). In none of these cases is the gas flow used as a carrier medium for liquids or other gases. Furthermore, no Venturi nozzles are used at the outlet opening, which intensify the mixture with the reaction liquid and thus the effectivity of the aeration.

### Liquid Dosage:

### a) Pumps

As a standard, pumps of any design are used for the quantitative dosage of liquids. They take an aliquot according to the setting of a superimposed regulator from a storage vessel and pump it through a supply line to the reaction vessel. The transporting force constitutes the pump capacity. For biological or biochemical culture vessels, dosage pumps for acid, lye, anti-foam agent and one to two substrate solutions are typical, and these simply pump the liquid into the reaction liquid (Braun Biotech International GmbH, bio-reactors series BIOSTAR A, B, MD, Q, D, U). In none of these cases is the liquid contacted with a gas flow leading to an aerosol generation and thus to a homogeneous mixture and to a more efficient use of the gas.

### b) Pressure Feeds

For larger culture vessels (more than approx. 50 liters), liquid feed vessels are used, which have an overpressure compared to the culture vessel and are connected therewith by a supply line with an integrated clock valve. If a liquid dosage is to take place, a regulator opens the clock valve for a certain time, so that by the overpressure liquid is pressed to the culture vessel. By means of the parameters of opening time, the cross-section of the supply line, overpressure and viscosity of the liquid, the dosage can quantitatively be calibrated (Braun Biotech International GmbH, bio-reactors series customer-specific production systems). For biological or biochemical culture vessels, feed vessels for acid, lye, anti-foam agent and one to two substrate solutions are usual, which simply “press” the liquid into the reaction liquid. In none of these cases is the liquid contacted with a gas flow leading to an aerosol generation and thus to a homogeneous mixture of more efficient use of the gas.

c) Mixing Stations with Venturi Nozzles

Venturi nozzles as such are known from fields other than bioreactors. Due to their flow characteristics, Venturi nozzles generate an underpressure at the side inlet, allowing another medium 2 (gas or liquid) to be sucked in toward the flowing medium 1 (gas or liquid). In the outlet section of the nozzle, a homogeneous mixture of the two media takes place. If the cross sections of the nozzle, the viscosity of the media and the inlet pressure of the nozzle are known, a quantitative mixture can be achieved. Medium 1 may continue functioning behind the nozzle due to its overpressure as a transport medium.

Venturi nozzles are used for manifold applications for aeration (water-jet pumps), in flowmeters (delta pressure) or for the mixture of various media, e.g. dilution of concentrates with a second medium. On a micro scale, Venturi nozzles can be employed for a quantitative sampling of a medium (Fox Valve Development Corp., Hamitton Business Park, Dover, N.J. 07801 USA, Internetfoxyalve.com). Although a multitude of applications for dosage and mixture in daily use are known for Venturi nozzles (e.g. Jacuzzi), and they do not have any movable wear parts and thus represent an ideal dosing device, the application of such nozzles in the sector of biological and biochemical culture vessels for the dosage of gases or liquids by means of a transport medium has not been described before.

Furthermore, no dosing system according to the present invention for biological and biochemical culture vessels has been described, which can combine a transport medium with several dosage media (gas or liquid), permits a quantitative dosage and in addition, if applicable, has atomization nozzles or mixing nozzles at the outlet, in order to secure a better mixture with the reaction liquid or a more effective use of the dosed liquid.

As a summary, the disadvantages of the prior art for culture vessels for biological and biochemical culture vessels are:

Not suitable for a miniaturization below 1,000 ml culture vessel volume.

Expensive for parallel systems, susceptible to interferences, uneconomical, and only of limited utility use.

High cost.

The dosage of liquids, gases or mixtures thereof into culture vessels for biological and biochemical reactions requires enormous economic and mechanical efforts and is not reasonable for a larger number of culture vessels operated in parallel.

### TECHNICAL OBJECT OF THE INVENTION

It is therefore the object of the invention to provide an effective, economical and miniaturization-suitable fluid dosing method as well as a device therefor.

### BASICS OF THE INVENTION

The object is achieved by a method and a device for producing a carrier fluid, which can simultaneously be used for the aeration of the culture vessel.

To the carrier fluid can be quantitatively admixed the fluids to be dosed. Without the use of pumps and other complicated mechanical parts, defined conditions can in this way be established in the culture vessel in the reaction liquid and in the atmosphere of the vessel, and simultaneously the properties of the dosed fluid are utilized in an optimum manner.

The invention is particularly suited for the parallel operation of several culture vessels. The present invention can be used in all fields where biological or biochemical reactions are performed in culture vessels, particularly in the biotechnology, food technology and environmental protection fields.

### BRIEF DESCRIPTION OF THE FIGURES

FIG. 1 shows a Transport Medium Gas device and the method of producing a carrier fluid.

FIG. 2 shows a Transport Medium Liquid device and the method of producing a carrier fluid.

FIG. 3 shows a Dosage Feed for Gases, Transport Medium Gas device and the method of producing a carrier fluid

FIG. 4 shows a Dosage Feed for Gases, Transport Medium Liquid and the method of producing a carrier fluid

### DETAILED DESCRIPTION OF THE INVENTION

The above object is achieved, with regard to the method, in that the fluid to be dosed or the fluids to be dosed are admixed in a defined concentration to one or several carrier and transport fluids (carrier fluids), and that this carrier fluid or these carrier fluids, respectively, are supplied in a defined amount and/or time units to the culture vessel either into the reaction medium or into the headspace of the vessel.

The above object is achieved, with regard to the device, by devices for admixing one or several fluids to be dosed to one or several carrier fluids, and supplying them to one or several culture vessels, as described in the following examples and the patent claims.

In the following the description of the individual modules and properties according to the invention is given with reference to a 1,000 ml culture vessel with 500 ml liquid volume. It is specifically emphasized that the numbers (in particular the relative statements) can be adjusted to culture vessels having volumes of 1 ml to 50 m³, the cross sections of the nozzles and dosing sections respectively having to be adjusted.

### a) Gas as the Transport Medium of the Device

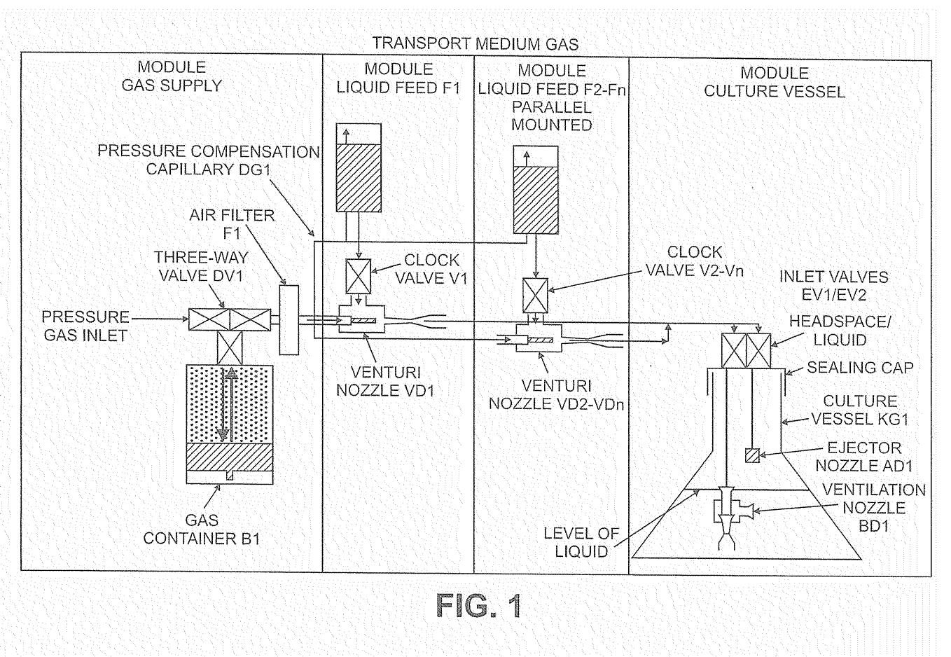

The module gas supply of the device is composed of the following essential components (FIG. 1):

Pressure gas inlet.

Three-way valve DV1 or inlet and outlet valve.

Gas container B1.

Gas filter F1.

Pressure compensation duct DG1.

The pressure gas inlet with an input over-pressure compared to the culture vessel of 0.1 to 10 bars, preferably 0.2 to 1 bar, in particular 0.5 bar, is connected via a pressure-resistant hose, having an internal diameter of 0.5 to 8 mm, preferably 0.5 to 2 mm, in particular 1 mm, to the three-way valve DV1 (see FIG. 1). The valve DV1 is arranged such that the gas container B1 with a container volume of 1 to 40%, preferably 1 to 10%, in particular 5%, of the liquid volume in the culture vessel, is filled up with pressurized air or another gas. A built-in piston can vary the filling volume of the gas container from 0 to 100% of the container volume. After achieving the pressure compensation, the valve DV1 is changed to the other position, gas container-culture vessel. By the pressure compensation, a gas flow toward the culture vessel is generated, and said gas flow can be conducted behind an optional gas filter through the modules described below and finally flows out in the headspace or the reaction liquid of the culture vessel. The pressure compensation capillary branching off behind the three-way valve DV1 provides for an equalized pressure between the gas supply and the modules liquid feed. At the output of the module gas supply, a filter may be provided for the filtration of the transport medium. The culture vessel is supplied by this device according to the invention discontinuously in a simple way with defined and thus quantifiable “gas portions”. The smaller the container volume and the higher the clock rate of the valve, the more this discontinuous gas flow comes closer to a continuous gas flow. In the following table, the container volume is 5% of the liquid volume of the reaction liquid (example 25 ml container volume, 500 ml reaction liquid volume) and the aeration rate VF is the quotient of gas volume/h divided by volume of reaction liquid.

TABLE 1

| Clock rate | Gas flow/min | VF |
|---|---|---|
| valve/min | in % liquid volume | (l/h) |
| 0 | 0 | 0 |
| 1 | 5% | 3 |
| 2 | 10% | 6 |
| 5 | 25% | 15 |
| 10 | 50% | 30 |
| 15 | 75% | 45 |
| 20 | 100% | 60 |
| 25 | 125% | 75 |
| 50 | 250% | 150 |

For aerobic, biological or bio chemical reactions, the VF values are usually between 5 and 60 (l/h). This can easily be achieved with the present module according to the invention in a nearly “continuous” gas flow, complicated mechanical or electronic flow measurements and regulators not being required. Essential for an optimum and continuous gas supply of cultures of microorganisms with optimum use of the gas is the so-called “gas hold-up”, i.e. the hold-up time of the gas bubbles in the reaction solution, whereas the gas exchange can take place at the interface between gas bubble and liquid by diffusion. An optimum use of the gas with simultaneous optimum aeration rate is achieved, when the “gas hold-up” is equal to the clock rate of the valve DV1. There is always a dosage of gas when the gas bubbles disappear from the liquid. A variation of the amount of passed-through gas can be achieved by the variable volume of the gas container. Furthermore, the structure according to the invention of the module reduces the tendency for foam generation, since there is dosed always that amount only of gas, which is necessary for an optimum supply to the culture.

### b) Liquid as the Transport Medium

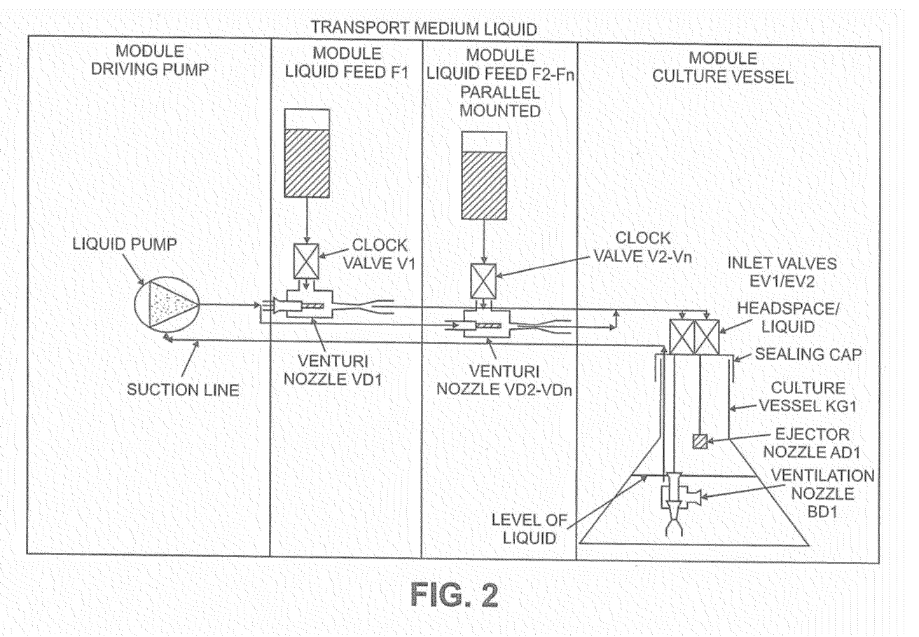

In lieu of the module gas supply, liquid can be used as the transport medium. In this case, the module gas supply is replaced by a controlled liquid pump, which is either connected by a suction line to the reaction liquid in the culture vessel and circulates the liquid or sucks it in from its own storage vessel (FIG. 2). The module driving pump is composed of the following essential components:

Liquid pump.

Suction line.

Pressure line toward the culture vessel.

Filter (optional).

The use of liquid as the transport medium is then particularly useful, if the reaction liquid is to be enriched efficiently, while avoiding gas bubbles in the culture with gases, e.g. CO₂ dosage in cell culture media or dosage of minimum amounts of substances. The dosage of catalyzers or the dosage of biological active ingredients can, for instance, be mentioned here. Active ingredients are in most cases extremely expensive and are stable for long times in a concentrated form only. According to the invention, they are dosed with liquid modules (see below) in smallest amounts and in arbitrary combinations.

### c) Module Liquid Feed

The module liquid feed is composed of the following essential components (FIG. 1):

Storage container liquid.

Pressure compensation line, branched-off from the pressure compensation capillary.

Supply line to the clock valve and the Venturi nozzle.

Clock valve.

Venturi nozzle.

The liquid feed is filled with a liquid to be dosed to the reaction liquid in the culture vessel, and a remaining volume of gas of at least 2% of the volume of the feed must be present for the pressure compensation. If the transport medium is a liquid, there need not be provided the remaining volume of the gas and the pressure compensation by capillaries (FIG. 2). Instead, the feed can be aerated with atmospheric external pressure to prevent underpressurization. The liquid feed can be installed in any position, suspended, standing, lying with regard to the device, and the pressure compensation line should terminate in the present gas volume. The liquid feed has, compared to the liquid volume of the reaction liquid, a volume of 0.5 to 50%, preferably 5%. It is connected by a line to the clock valve V1, and the latter to the Venturi nozzle VD1. If the module gas supply or driving pump delivers a flow of transport medium via the Venturi nozzle, at the side inlet of the nozzle, an underpressure will be generated, compared to the otherwise pressure-compensated system. With simultaneous opening of the clock valve V1, liquid is sucked in from the liquid feed toward the gas flow in the nozzle. The sucked-in amount of liquid correlates with the following parameters and can therefore be quantitatively and reproducibly calibrated. Therefore, it is possible to perform a quantitative dosage of liquid aliquots to the transport medium based on the cycle time of the valve V1 only at constant parameters according to Table 2.

TABLE 2

Nozzle dimensions.
Pressure and gas flow through the nozzle.
Cross-sections of the supply line and of the clock valve.
Viscosity of the liquid.
Temperature.

In the outlet of the nozzle, the sucked-in liquid and the transport medium are homogeneously mixed. Between the module gas supply or module drive (FIGS. 1 and 2) and the module culture vessel, several modules of liquid feed, preferably 4 modules, can be installed. The installation can be parallel (preferred) or in series. In this way it is possible to quantitatively dose into the transport medium simultaneously from no liquid to several different liquids, to combine them in any amounts and to homogeneously mix them before the inlet into the culture vessel. In biological cultures, beside the titration of the pH value with acids and bases and the addition of means for foam abatement, in particular so-called “fed batch” methods are usual. Herein, one or several substrates, e.g. carbon or nitrogen source, are dosed to the culture in a controlled manner. The present device permits in a very simple way to vary the composition of the liquid dosage. For instance, by the variation of the cycle time only, substrate gradients can be established in dependence of the time or of culture-specific control parameters, or additional nutrients can be admixed, such as growth factors, minerals or vitamins from further liquid modules.

### d) Module Dosage Feed for Gases

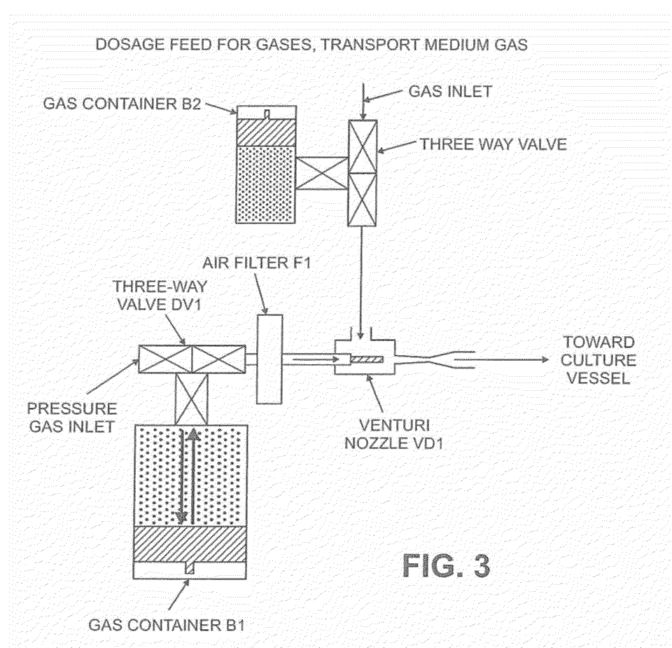

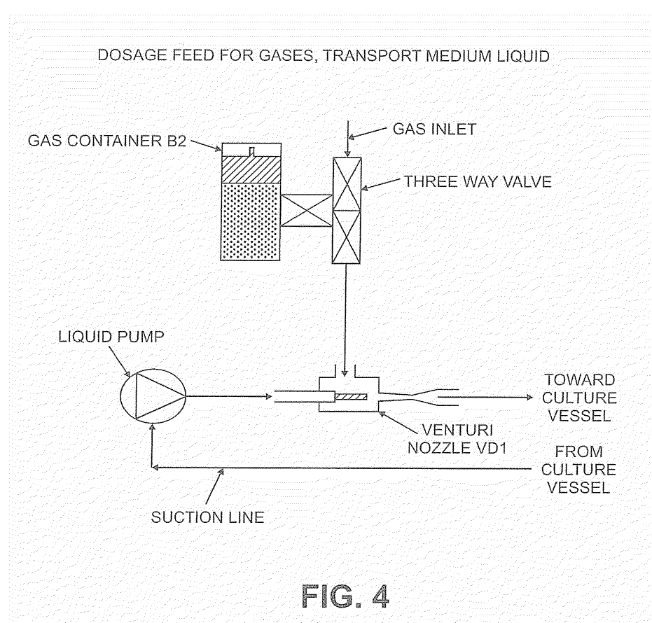

The module dosage feed for gases (FIGS. 3 and 4) is composed of the following essential components:

Gas container B2, adjustable by a piston in the filling volume.

Gas inlet.

Three-way valve.

The three-way valve is installed between the gas inlet and the gas container B2. The container fills up with gas, and the filling volume can be varied by the built-in piston, and is, however, quantitatively known. If now a gas dosage is to be made, the three-way valve is switched over for a defined cycle time toward the Venturi nozzle, and it should be made sure that there is an underpressure at the nozzle generated by the transport medium. With known inlet pressure at the gas inlet, filling volume of the gas container and cycle time of the three-way valve, a quantitative gas dosage can be achieved. Between the module gas supply or module drive (FIGS. 1 and 2) and the module culture vessel, several modules' gas dosage, preferably for 2 modules, can be installed. The installation can be parallel (preferred) or in series. In this way it is possible to quantitatively dose into the transport medium simultaneously from no gas to several different gases, to combine them in any amounts and to homogeneously mix them before the inlet into the culture vessel. The gas modules can be used in lieu of or in any combination with the liquid modules. In biological cell cultures, frequently CO₂ is employed for regulating the pH value, which can easily and quantitatively be dosed with this module while avoiding gas bubbles in the reaction liquid. Furthermore, by the gas dosage, an artificial atmosphere can be created and controlled in the culture vessel, which is advantageous for biological cultures. For instance, the culture of plant cells prefers a higher CO₂ concentration (as a substrate), or the breeding of anaerobic organisms in a nitrogen or sulfur atmosphere.

### e) Module Culture Vessel

The module culture vessel is essentially composed of the following components:

Culture vessel KG1, filled with the reaction liquid and gas space thereabove (headspace) and cover of the vessel.

Supply line for the transport medium.

Inlet valve EV1 with supply line into the headspace of the culture vessel.

Inlet valve EV2 with supply line into the reaction liquid. Ventilation nozzle BD1 in the reaction liquid.

Ejector nozzle AD1 in the headspace of the culture vessel.

By the inlet valves provided at the cover of the culture vessel, it is possible to select whether the transport medium is to be dosed into the air space of the culture vessel (“headspace”) or into the reaction liquid. The inlet valve EV1 to the headspace leads to an atomization nozzle AD1 installed in the air space, which again generates an atomization of the transport medium. The complete, atomized transport medium and the dosages go uniformly down onto the surface of the reaction liquid. This fine distribution causes a quick mixture of the transport medium and the dosages with the reaction liquid and can lead to a more efficient use of the dosed liquid. The efficiency of anti-foam agents, which are dosed in this way, can hereby be increased by 10 times, thus the consumption can correspondingly be minimized. Further, it is possible to use a gas flow only without dosed liquid for foam abatement. The foam is simply “blown down” by the gas flow. Frequently, this effect is already sufficient for foam abatement, without the necessity of subsequently dosing anti-foam agents as described above. The avoidance of anti-foam agents in biological processes is the ultimate goal, since they could have negative effects on the culture itself and on the later purification process, and are biologically poorly degradable and can therefore not easily be disposed of.

Headspace dosages in the above manner are mainly used when an aeration of the surface of the reaction liquid only is desired, e.g. for anaerobic cultures or if liquids are dosed, which should have a fast effect on the reaction liquid. As an example, the titration of the pH value with acids or bases, and foam abatement in the manner described above. The inlet valve EV2 leads to a Venturi nozzle BD1 arranged in the reaction liquid. The transport medium (and the dosages) flows through the ventilation nozzle BD1 into the reaction liquid. Reaction liquid is sucked in at the side inlet of the nozzle because of the generated underpressure, said reaction liquid being effectively mixed in the outlet section of the nozzle. If the microorganisms (e.g. tissue cells) are not to be subjected to the shearing forces in the nozzle, the side inlet opening can be sealed by a filter membrane. In addition to the mixing effect drastically reducing the mixing times of the reaction liquid, and to the clearly accelerated gas exchange rates, many more smaller air bubbles are generated (with trans-port medium gas) than with prior art aerations. These smaller bubbles increase the border area available for the gas exchange between air bubble and reaction liquid, that is, they increase the gas exchange rate and remain for a longer time in the reaction liquid than large bubbles, thus increase the “gas hold-up” and therefore again the gas exchange rate. The gas is used in a more effective way, so that, if applicable, and depending upon the kind of cultivation, shaking or stirring of the culture vessel is not necessary. Furthermore, the tendency for foam formation is minimized by smaller bubbles. If aerosols are dosed in this way, e.g. substrates in the gas flow, the shorter mixing times will lead to a faster, homogeneous distribution in the reaction liquid. Substrate gradients caused by poor mixing can be prevented, and the culture is uniformly supplied in the desired manner.

The present invention has the advantage that it combines in a suitable way function modules for a completely new field of application and thus unites a previously expensive and complex technology with a simple, compact device. The use of the device for biotechnical processes under sterile conditions becomes possible, and control functions become available to new fields, which up to now could not be solved by prior art devices. As an example is mentioned here the novel parallel fermentation of culture vessels, usually up to 16 vessels (Das GIP GmbH, www.dasgip.de), for the optimization of media and processes for biological methods. Herein, the effects of different parameters on the result of the culture are intended to be investigated under near-production conditions, and with regard to measurement and control, the conditions of the production facility would already be desirable as far as possible, i.e. effective aeration and dosage of different liquids. As already mentioned above, such a parallel fermentation would require 96 pumps, 96 regulators and 16 controlled supply sections, and is therefore technically and economically impractical. Nonetheless, such fermentation does not meet, even when bubble columns are used as the culture vessel, the measurement and control conditions of a production facility.

The trend is to a further miniaturization and increase of the number of culture vessels, in order to obtain in a shorter time more results in a reproducible and quantifiable form (recordable). This is not achievable anymore with prior art devices, but may be achieved, however, by means of the present invention. The function modules can be produced in any size and can thus be adjusted to the size of the culture vessel, and the volumes of the culture vessels may be between 1 ml and 50 cubic meters. For culture vessels having a liquid volume of 1 ml to 500 ml, the complete device including the liquid and gas feeds and the valve controller can be fixed at the neck of the culture vessel. The data exchange with the control EDP system takes place via an infrared interface. There is thus only one supply line to the culture vessel required, consisting of a gas supply line and a power supply. A further miniaturization of the device can take place in that the functional parts and supply lines are etched, cut or molded in corresponding materials, such as steel and plastic materials, and the valve function is achieved by inserted seals operated by pistons, or arbitrary other mini-valves. The device according to the invention can be combined with constructs in the culture vessel, e.g. patent application having the title “Device as construct for culture vessels for optimized aeration and dosage of shaken or stirred three-phase systems” (file number will be submitted later). By the combination, a high-performance culture vessel is generated, which reproduces and can simulate in a very simple way in a nearly arbitrary scale the complete measurement and control technology and the process parameters of a high-performance fermenter.

### Example of Execution

### Materials

Culture vessel: 1,000 ml Erlenmeyer flask (narrow neck) with Kapsenberg.

### Composition of the Medium:

| | | |
|---|---|---|
| Yeast extract for the microbiology | 20 | g/l |
| Glucose for the microbiology | 1 | g/l |
| Ammonium sulfate | 1.5 | g/l |
| Common salt | 0.1 | molar |
| Magnesium chloride | 0.5 | g/l |
| Potassium phosphate buffer | 0.1 | molar, pH 7.2 as solvent |
| Olive oil, extra virgin | 1 | ml/l |

Three-way valve DV1: The Lee Company, type LHDA12311115H. Clock valve V1, V2: The Lee Company, type LFVA 1230210H.
Inlet valve EV1, EV2: The Lee Company, type LFVA 1230210H.
Venturi nozzle VD1, VD2: Spraying Systems, type.
Ventilation nozzle BD1: Spraying Systems, type.
Ejector nozzle AD1: Spraying Systems, type.
Air container B1: Braun Melsungen, disposable syringe 50 ml with Luer Lock.
Air filter F1: Sartorius, disposable sterile filter, 0.2 μm.
Liquid feeds: disposable ampules, 25 ml with flange cap and rubber seal.
Hoses: Teflon hose, 1 mm inner diameter.

### Couplings: Luer Lock.

Foam-detection: isolated needle with mass connection to the reaction liquid.
Valve controller: Braun Melsungen DCU 3 system.

The components of the medium are obtainable from typical specialty sources specialist shops in identical quality. The components, glucose and magnesium chloride, are separately sterilized as suitable aliquots and then added under sterile conditions. The culture vessel was filled with 500 ml medium and sterilized in the autoclave. The supply lines to the headspace and to the reaction liquid with the nozzles were guided through a bore in the cover, sealed and equally sterilized together with the vessel. The separation to the device according to the invention was made at the exit of the inlet valves. Liquid feeds served 24 ml glucose solution (100 g/l) and 24 ml anti-foam agent (Dow silicon oil, 10% suspension) each, which were separately sterilized. The device according to the invention was installed, as far as there were no other fixing means provided for the individual components, according to FIG. 1 with Luer Lock fittings and Teflon hoses and fixed on a working panel. The power part between the air filter exit and the exit of the outlet valves as well as the supply and discharge lines of the liquid feed are decontaminated with 10 m soda lye (2 h), and then rinsed with sterile 0.1 m phosphate buffer pH 7.2.

After the sterilization and cooling-down of the culture vessel, the inoculation was performed with a pure culture of the microorganism with one milliliter each under sterile conditions. The pure culture was produced from a tube E. coli, K12, obtainable from the German culture collection (DSM Hannover), and the contents of this tube were cultivated in 10 ml standard 1 medium (Merck Darmstadt) at 37° C. for over 12 hours under sterile conditions. The optical density of the pure culture was at the time of the inoculation 0.9 OD (546 nm). The device was coupled with the inlet valves to the culture vessel and to the module gas supply. To the liquid feed 1 was connected the glucose solution, to the second one the anti-foam agent. The liquid feeds were used in a standing orientation. As a connection for the pressure superimposition, a short disposable injection needle as used, for the liquid removal a long one, which was passed through the rubber seal in a sterile manner. At the pressure air inlet, pressurized air with an overpressure of 0.5 bar was connected. The volume of the gas container was adjusted to 25 ml. The complete device and the culture vessel were tempered to 37° C. in an incubator. The culture vessel was not shaken, since the gas flow alone provided for a sufficient gas supply of the culture. The valves of the device according to the invention were connected to the control unit DCU3 and regulated, as shown in Table 3:

TABLE 3
Gas supply:
Clock rate of 15 fillings and gas flows per minute, corresponds to a
VF of 45 or 22.5 l air/h, inlet valve EV1 closed, EV2 open, i.e. gas flow
into the reaction liquid.
Liquid feed 1, substrate:
Clock valve V1, opened four times per minute for 0.2 seconds, at
the same time as the connection of a gas flow to the culture vessel, DV1
open toward the culture vessel, EV2 open, corresponds to a glucose
dosage of 1 mi per hour.
Liquid feed 2, anti-foam agent:
Clock valve V2 normally closed. When the transducer needle
indicates a foam signal, the following algorithm proceeds: inlet valve EV2
is closed, inlet valve EV1 opened, i.e. headspace aeration start of a
timer. If the foam signal of the transducer needle is negative after 8
seconds, the valve EV1 is closed, and the valve EV2 opened, return to
standard operation. If the foam signal continues to be present, then at the
same time as every clock signal of the gas supply, the clock valve V2 is
opened for 1 second, and so anti-foam agent (18.7 ml/h) is admixed to the
air flow of the gas supply. If after another 16 seconds the foam signal is
still present, the valve EV2 is in addition opened, in order to supply gas
to the culture again. This condition is maintained, until the signal of the
transducer needle is negative. Then return to standard operation.

After 24 hours, the cultivation of the microorganisms was stopped, and the optical density (OD) was determined at 546 nm with a photometer. The OD of approx. 90 corresponds to the value to be expected in a high-performance fermenter and demonstrated the capabilities of the device. The substrate feed was completely consumed at this point of time. For the anti-foam agent was measured a consumption of approx. 2 ml, distinctly less than the amount, which a conventional fermenter would have needed for this result (approx. 12 ml, depending upon the regulation algorithm).

During the execution of this example, the following could clearly be observed in particular:

The compact, simple type of execution of the device according to the invention.

The effectivity of the “pulsed” aeration system in combination with the ventilating nozzle.

The generated extremely fine gas bubbles.

The short mixing times of the system.

The performance of the foam abatement by the structure according to the invention.

The precise uniform dosage of the liquids.

Once again it is emphasized that these results, which correspond to those of a high-performance fermenter, were achieved without shaking or stirring. In combinations with inserts, by shakers or stirrers, the performance can further be increased.

1. A bio-reactor for the cultivation of cells, comprising a culture vessel, one or several gas supplies and/or one or several liquid supplies as well as supply devices for gases and/or liquids, by means of which gases and/or liquids can be added to the culture vessel, wherein between the supply device and the gas supply or liquid supply, a mixing device comprising a Venturi nozzle, for mixing gas or liquid from the gas supply or the liquid feed is installed.

2. A bio-reactor according to claim 1, wherein gas and liquid are mixed to an aerosol.

3. A bio-reactor according to claim 1, wherein between the mixing device and the gas supply or liquid feed, controllable valves comprising clock valves, are installed.

4. The bio-reactor according to claim 1, wherein the gas supply comprises at least one gas supply module according to drawing FIG. 1 and the bio-reactor further comprises a driving pump module according to drawing FIG. 2 for producing a carrier fluid, a Venturi nozzle with clock valve and at least one liquid supply module with a pressure compensation capillary, or a dosage feed for gases, wherein the supply device terminates in the reaction liquid or in a headspace of the culture vessel, and the gas supply module or the driving pump module generates a flow of carrier fluid, continuously or discontinuously, through a supply line toward the culture vessel.

5. The bio-reactor according to claim 1, wherein the gas occupies a volume between 1 and 40% of the volume of a total volume of the culture vessel.

6. The bio-reactor according to claim 1, wherein the gas occupies a volume in the range of 0 to 100% of the total volume of the culture vessel and the volume is varied by a piston in the range of 0 to 100% of the total volume.

7. The bio-reactor according to claim 4, wherein the clock valve has a clock rate that is adjustable to up to a limit comprising the “gas hold-up” of the gas bubbles in the culture vessel, preferably is identical thereto, and the amount of the passed-through gas is varied within a range of 0 to 100% of the total volume of the culture vessel by a piston.

8. The bio-reactor according to claim 4, wherein at least one liquid supply module or gas supply module according to drawing FIG. 1 is installed between the valve and the culture vessel.

9. The bio-reactor according to claim 4, wherein the liquid supply module is comprised of at least one Venturi nozzle and a liquid container and pressure compensation takes place either through the pressure compensation capillary to the gas container or through a connection to the external atmosphere.

10. The bio-reactor according to claim 9, wherein the liquid supply modules are installed in any position, suspended, standing, lying with respect to the culture vessel which has an air space of at least 2% of the total volume of the culture vessel in which the pressure compensation can take place.

11. The bio-reactor according to claim 4, wherein the gas dosage feed is comprised of at least one gas inlet, one three-way valve or two valves, a gas container with variable internal volume or a valve opening connected to a side inlet of a Venturi nozzle.

12. The bio-reactor according to claim 4, wherein dimensions of components of the bio-reactor are adjustable to culture vessels from 1 milliliter to 50 liters volume.

13. The bio-reactor according to claim 4, wherein the bio-reactor further comprises an atomization device comprising an ejector nozzle at an entrance of a headspace of the culture vessel.

14. The bio-reactor according to claim 4, wherein formation of foam in the reaction liquid is reduced by discontinuous aeration and the gas supply.

15. The bio-reactor of claim 4, wherein gases and liquids dosed in the reaction liquid or in the headspace of the culture vessel results in a reduced mixing time and minimization of concentration gradients comprising a concentration gradient of a substrate within the culture vessel.

16. The bio-reactor of claim 4, wherein the dosage feed into the headspace of the culture vessel results in an effective use of the properties of the dosed liquids takes place.

17. The bio-reactor of claim 4, wherein the use of anti-foam agents is minimized.

18. The bio-reactor of claim 4, wherein dosed liquids or a combination of several of them can have an effect on the reaction liquid.

19. The bio-reactor of claim 4, wherein dosed gases are used for generating an artificial atmosphere in the headspace of the culture vessel.

20. The bio-reactor of claim 4, wherein the bio-reactor is connected to a power supply and transport medium supply, and all measurement and control parameters are exchanged with a control EDP system via an infrared interface.
